(19) United States
(12) Patent Application Publication
(10) Pub. No.: US 2007/0249007 A1
(43) Pub. Date: Oct. 25, 2007

(54) METHOD AND APPARATUS FOR THE MANAGEMENT OF DIABETES

(76) Inventor: Spencer Z. Rosero, Pittsford, NY (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 11/738,271

(22) Filed: Apr. 20, 2007

Related U.S. Application Data
(60) Provisional application No. 60/794,217, filed on Apr. 20, 2006.

(51) Int. Cl. C12Q 1/00; C12Q 1/54; C12M 3/00

(52) U.S. Cl. 435/14; 435/287.1; 205/777.5

(57) ABSTRACT

A biological chip device having a biologic materials component according to embodiments of the invention may provide the ability to sense levels of blood parameters (e.g., glucose, insulin, and glucagon levels) in a patient. In certain embodiments of the invention, a device may include therapeutic abilities in which a portion of the biologic materials component produces a drug to treat the underlying disease in response to measured or sensed blood parameter levels.

### FIELD OF THE INVENTION

Embodiments of the invention relate generally to an apparatus, system and method for using biologically based devices to monitor and treat disorders such as diabetes.

### BACKGROUND SECTION

Diabetes is a disease that affects the body's production of insulin, a hormone used to control how a body's cells handle blood sugar levels and other nutrients to provide energy for the cells. Diabetes can be caused by a variety of factors, many of which are poorly understood but appear to be influenced by genetics (i.e., a family history of diabetes), and factors such as obesity and lack of exercise.

It is estimated that there are over 20 million people in the United States, or roughly 7% of the population, who have diabetes. Diabetes is typically classified according to its “type.” For example, Type 1 diabetes results from the body's failure to produce insulin, the hormone that “unlocks” the cells of the body to allow glucose to enter and fuel the cells. It is estimated that 5-10% of Americans who are diagnosed with diabetes have Type 1 diabetes.

Type 2 diabetes results from insulin resistance, a condition in which the body fails to properly use insulin, combined with relative insulin deficiency. Most Americans who are diagnosed with diabetes have Type 2 diabetes.

Gestational diabetes affects about 4% of all pregnant women. There is estimated to be about 135,000 cases of gestational diabetes in the United States each year.

Pre-diabetes is a condition that occurs when a person's blood glucose levels are higher than normal, but not high enough for a diagnosis of Type 2 diabetes. There are over 40 million Americans who have pre-diabetes, in addition to those with diabetes.

In order to determine whether or not a patient has pre-diabetes or diabetes, health care providers conduct a Fasting Plasma Glucose Test (FPG) or an Oral Glucose Tolerance Test (OGTT). Either test can be used to diagnose pre-diabetes or diabetes.

With the FPG test, a fasting blood glucose level between 100 and 125 mg/dl may indicate pre-diabetes. A person with a fasting blood glucose level of 126 mg/dl or higher has diabetes.

In the OGTT test, a person's blood glucose level is measured after a period of fasting, and two hours after drinking a glucose-rich beverage. If the two-hour blood glucose level is between 140 and 199 mg/dl, the person tested has pre-diabetes. If the two-hour blood glucose level is at 200 mg/dl or higher, the person tested has diabetes.

Current methods of treating diabetes patients typically include performing one or more of the following steps: 1) measuring a blood glucose level; 2) calculating an amount of insulin to be delivered to the patient; and 3) delivering the insulin. Delivery of insulin may occur via a number of known methods, such as manual injections (e.g., using a syringe), delivery by an insulin pump, and other forms of insulin that may be inhaled or ingested, for example.

Determining the amount of insulin to be delivered may be prone to certain errors. For example, erroneous blood glucose level readings may occur due to technical issues with the measurement, or due to human error, or both. Similarly, calculating the amount of insulin to be delivered may be prone to both human and/or technological errors. Further, the amount of insulin actually (or effectively) delivered to a patient may, in some cases, be different from the amount calculated, possibly due to errors or malfunctions in the delivery system, for example.

A method of determining a treatment (e.g., an amount of insulin to be delivered to a diabetes patient) based upon a physiologic need (e.g., a blood glucose level in a diabetes patient) is therefore desired which reduces the potential for errors, and which may produce a more physiologic result.

### SUMMARY OF INVENTION

Recent advances in molecular medicine, pharmaceutics, and electronics have allowed the integration of biological materials with electronics on a common platform. The integration of living tissue and individual cells with electronic and non-electronic based devices may find applicability in a number of medical device technologies, for example. Such medical devices may include a biologic materials component sustained in a support housing or support matrix. A biologic materials component may consist of cells or tissue of interest (e.g., cardiac cells, vascular tissue, etc.), which may be obtained from a donor and/or a patient, and sustained and/or grown in a complex collagen or other biocompatible support matrix. The support housing or support matrix may be equipped with sensing electrodes adapted to sense/measure various parameters affecting the housing (e.g., environmental parameters) such as acceleration, pressure, flow, temperature, strain/shear stress, electrical signals, and electromagnetic signals, for example without limitation. An electronic component may also be included to facilitate information processing and/or communications with the biologic materials component.

The treatment of endocrinologic/blood disorders may be improved by the use of biologic based sensors to monitor certain biological parameters. Examples of biological parameters include blood parameter levels such as glucose, pH, and hormones such as insulin, and glucagons, for example without limitation. The use of a biologic based sensor may improve the specificity and sensitivity of the measurements and may thereby provide a more physiologic result. The biologic materials component (e.g., the cells or tissue) used in various embodiments of the invention may be determined by the type of biological parameter to be detected or sensed and the desired output or response. Multiple cells and or types of cells may be used to coordinate various inputs and outputs. The biologic materials component may respond to a pre-determined biological parameter (e.g., glucose or other chemical level in the blood or tissue of the patient) in a useful or informative manner. The response may be detected by either: 1) other groups of biologic materials components that then perform a specific function; 2) an electronic based device (which could also include a chemical, optical, or mechanical aspect, for example); or 3) both. A biologic based sensor device may also function as a stand-alone group of cells adapted to detect and respond to a biologic parameter or process of interest without the use of electronic (or optical, chemical, mechanical) components.

Certain embodiments of the invention may provide the ability to respond to levels of blood parameters (e.g., glucose, insulin, and glucagon levels). The response of the biologic materials component (e.g., the cells or tissue) may be provided to: 1) a separate device that may or may not be implanted (e.g., an insulin pump or external glucose monitoring system); and/or 2) another component of the same integrated biological cell matrix consisting of an electronics/photonics component that may collect, analyze, and/or transmit data to other implantable devices, or trigger/stimulate or monitor the response of another biological cell matrix (e.g., another biological chip device) within the same implanted device, which may include different cell types or combinations, for example.

A biological chip device according to embodiments of the invention may have several distinct groups of cells or biologic materials components that may be adapted to communicate with each other. Communication between biologic materials components may be facilitated by using the cells' own natural signals (such as biochemical messengers, organic and inorganic substances, changes in cell shape/morphology, density, etc.), or using genetically engineered responses (such as the secretion of fluorescent proteins of specific wavelengths, or secretion of substances produced by the cells in pre-determined quantities, for example), to communicate or transmit information.

A biological chip device according to embodiments of the invention may incorporate therapeutic abilities in which a portion of the biologic materials component produces a drug to treat the underlying disease. In the case of diabetes, a biologic materials component may secrete insulin in response to levels of glucose. The amount of insulin secreted may provide a baseline level of insulin (e.g., relatively low concentrations) in a continuous or cyclic manner, for example, and may be used in conjunction with insulin that is externally administered to the patient. The device may have the ability to provide all of the insulin needed by some patients, according to certain embodiments of the invention.

A plurality of biological chip devices and associated support apparatuses can be supported and linked in a network, for example, to perform desired functions. Communication between devices can be accomplished via fluid flow between support apparatuses, by radio frequency, fiberoptic, and/or electrical signals, and possibly using blood as a communication medium, or by direct metallic conducting media (e.g., wires), or a combination of the above.

### BRIEF DESCRIPTION OF THE DRAWINGS

FIG. 1(a) is a partial cut-away schematic side view of a support housing for a biological chip device according to an embodiment of the invention;

FIG. 1(b) is a schematic plan view of a biological chip device support housing according to an embodiment of the invention;

FIG. 2 is a schematic perspective view of a support housing for a biological chip device according to an embodiment of the invention;

FIG. 3(a) is a schematic perspective view of a support container in accordance with an embodiment of the invention;

FIG. 3(b) is a schematic view of a support container having a vibration mount according to an embodiment of the invention;

FIG. 4(a) is a schematic top plan view showing a support container according to an embodiment of the invention;

FIGS. 4(b) and 4(c) show a support container according to an embodiment of the invention;

FIG. 5 is a perspective view of a biological chip device according to an embodiment of the invention;

FIG. 6 is a schematic view of fluidic channels in a biological chip device according to an embodiment of the invention; and

FIG. 7 is a flow chart describing a method of treating a bio-chemical disorder in accordance with an embodiment of the invention.

### DETAILED DESCRIPTION

The following detailed description should be read with reference to the drawings, in which like elements in different drawings are numbered identically. The drawings depict selected embodiments and are not intended to limit the scope of the invention. It will be understood that embodiments shown in the drawings and described below are merely for illustrative purposes, and are not intended to limit the scope of the invention as defined in the claims.

Embodiments of the invention include a biologic-based sensor that is adapted to generate a signal that varies with changes in the level of certain biological parameters. For example, a sensor according to an embodiment of the invention may generate a signal that is a function of one or more blood parameter levels. A blood parameter level may include, for example, glucose levels, pH levels, levels of glucagons, insulin, etc.

A biologic-based sensor includes a biologic materials component (e.g., cells or tissue) capable of producing a response to certain biological parameters. Examples of cells that may be used according to certain embodiments include pancreatic cells, stem cells, epithelial cells, vascular smooth muscle cells, liver cells, neurologic tissue, and circulating blood cells, for example without limitation.

The response to biological parameters may vary according to various embodiments of the invention, and may include the production of fluorescent protein, for example, or the production of other measurable responses. By measuring the response of a biologic materials component (e.g., the response of a group of cells) to a particular biological parameter or substance (rather than attempting to measure the biological parameter or substance directly, for example), a sensor signal may be generated that is less prone to error and more likely to produce a physiologic result. In one possible embodiment, for example, the biologic materials component may comprise cells that respond by producing a modified insulin-like gene (or protein) that may include a fluorescent protein. The use of certain cells, and their associated response, may provide an estimate of glucose levels in a patient with diabetes by measuring the fluorescent protein response, according to certain preferred embodiments of the invention.

Embodiments of the invention may include a support housing or support matrix capable of sustaining the cells. The support housing may include the ability to supply nutrients and fluids to the cells, the ability to transfer energy to and from the cells, and/or the ability to remove waste products from the housing. A support housing for a biological chip device is described in U.S. provisional patent application Ser. No. 60/740,564, which is hereby incorporated by reference in its entirety.

A support housing may comprise a biocompatible support matrix, which may include a complex collagen in some embodiments. The support matrix may have sensing electrodes (which may be micron-sized) adapted to sense various environmental parameters such as acceleration, pressure, flow, temperature, strain/shear stress and electrical discharge/signals.

The support housing may be of various shapes and/or sizes, and may be otherwise individualized for particular applications. The support housing may be coupled to an electronic component or circuit, for example a printed circuit board, that can generate signals and/or translate signals received by the support housing (e.g., communicated from the biologic materials component) to a predetermined format for processing and/or relaying to another biologic materials component or biological chip device. A plurality of biological chip devices can be linked together in a network as needed to perform desired functions and/or communicate desired information. Communication between such devices can be accomplished, for example without limitation, via radio frequency, fiberoptic, and electrical signals (e.g., analog electrical subcutaneous signaling), using an available fluid (e.g., blood) as a communication medium, either alone or in combination with direct metallic conducting media (e.g., wires).

A support housing according to certain embodiments of the invention may provide a device or system for the storage, support, and transportation of biological chip devices. A device or system in accordance with certain embodiments of the invention may provide the “life-support” (including, for example, the supply of nutrients, removal of waste, and maintenance of environmental conditions) required for each device, and can maintain the environment to allow the biological material to survive in various conditions. Environmental parameters that may be controlled include, but are not limited to, temperature, viscosity, pH, light (e.g., stimulation and/or protection via wavelength-specific filters), fluid cycling, gas levels (e.g., oxygen, nitrogen, etc.). Environmental parameters may be further controlled by the inclusion of filters for removal of waste products, compartments that provide (e.g., by eluting) nutrients such as glucose, energy components, etc., and algorithms for circadian control of various environmental parameters, such as temperature.

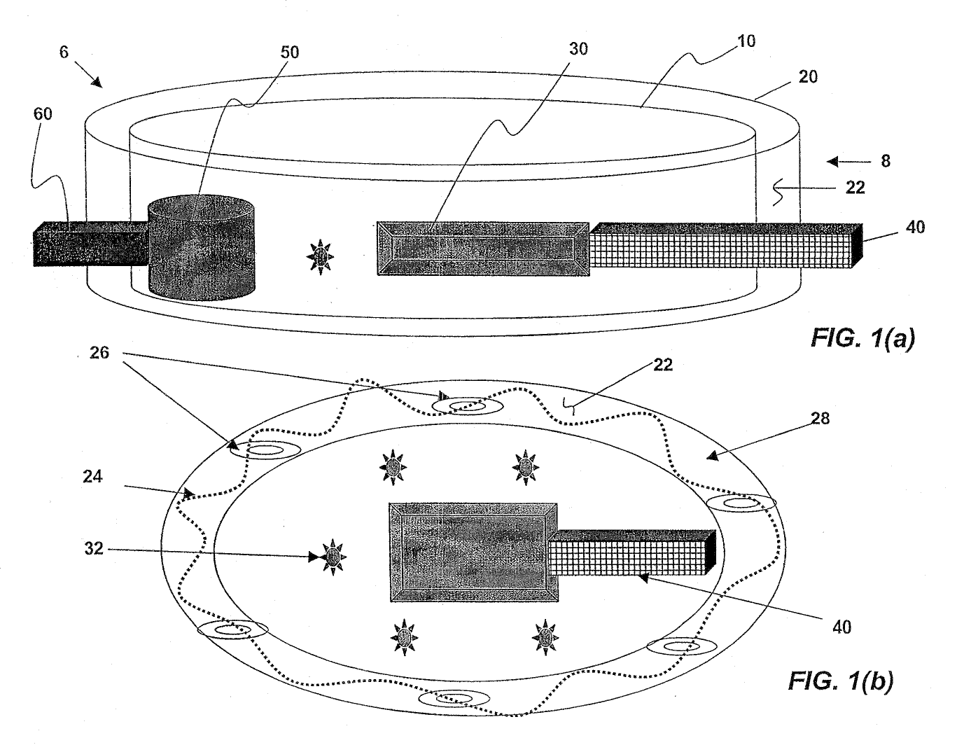

FIG. 1(a) shows a partial cut-away schematic side view of a support housing 6 for a biological chip device according to an embodiment of the invention. In the embodiment shown in FIG. 1(a), a support container 8 has an inner portion 10 and an outer portion 20. Inner portion 10 is disposed substantially within a volume defined by outer portion 20 such that a space 22 is formed between the inner and outer portions 10, 20. Inner and outer portions 10, 20 may be formed in a variety of shapes, such as generally cylindrical, generally rectangular or cubical, generally spherical, or any other shape suitable for a particular application. Further, the shape of inner portion 10 may be different from that of outer portion 20, and need not be centered within outer portion 20.

A biological chip device 30 is shown in FIG. 1(a) disposed within the inner portion 10, the biological chip device 30 being coupled to a connection strut 40. The connection strut 40 provides connections for providing one or more “services” to the biological chip device 30, such as providing an electrical power source, sending and/or receiving signals (i.e., electronic signals, electro-optical signals, signals in the form of electromagnetic energy, etc.). The connection strut 40 couples the biological chip device 30 in the inner portion 10 to a space outside the outer portion 20. This may be done, for example, by allowing the connection strut 40 to pass through the walls of the inner and outer portions 10, 20, as shown schematically in FIG. 1(a). Alternately, at least a portion of the services may be contained within the inner portion 10. For example, electromagnetic energy, such as radio frequency (RF) energy, may be communicated to a portion of the connection strut 40 situated within the inner portion 10. FIG. 1(a) also illustrates a fluid exchange 50 which may be disposed in the inner portion 10, with a fluid transfer conduit 60 that allows for the exchange (i.e., the delivery and/or removal) of gases and other fluids (e.g., oxygen, carbon dioxide, plasma, blood, nutrients, etc.) from outside the outer portion 20 into the inner portion 10, and vice versa.

FIG. 1(b) is a schematic plan view of the biological chip device support housing 6 of FIG. 1(a) according to an embodiment of the invention. In the embodiment shown in FIG. 1(b), a space 22 between the inner portion 10 and the outer portion 20 is shown having at least one thermal control element 24 (e.g., a heating element and/or a cooling element) disposed therein. In certain embodiments, one or more environmental sensors 26 may also be disposed within space 22. In certain preferred embodiments of the invention, a plurality of environmental sensors 26 may be disposed within the space 22 to monitor such environmental parameters as temperature, pressure, radiation (e.g., ultraviolet radiation), fluid/gas flow, pH, salinity, oxygen level, carbon dioxide, glucose, etc.

Environmental sensors 26 located within space 22 may be adapted to monitor and control the environment within the inner portion 10, but may also be able to respond to instructions (e.g., software-based instructions) for carrying out certain tasks. For example, diagnostic checks of the biological chip device 30 may be performed as a series of software-based instructions that may, for example, transiently lower the ambient temperature of the biological chip device 30 in order to obtain response data at various temperatures to compare with control (“normal”) values or to run calibration tests. The support housing 6 may be further adapted to release a chemical substance to the biological chip device 30 in response to certain instructions, for example, to trigger a cell response to perform a calibration check or to check the viability of the living cells.

In certain embodiments of the invention, the thermal element 24 may be adapted to automatically respond to certain of the environmental sensors, for example, by providing heating or cooling to the support container 8 when temperature in the space 22 reaches predefined setpoints. For example, thermal element 24 may comprise heating elements and/or cooling elements that may respond to control the temperature in the space 22 (and indirectly, the temperature within the inner portion 10) by alternately energizing and de-energizing the heating and/or cooling elements.

In certain embodiments of the invention, the space 22 between the inner portion 10 and the outer portion 20 may contain a fluid 28, such as a gel, to provide thermal insulation (i.e., to protect the inner portion 10 from changing environmental conditions outside the outer portion 20, for example), and/or to facilitate heat transfer (i.e., to raise or lower the temperature of the inner portion 10 via the thermal elements 24, for example).

The support container 8 of support housing 6 may include water-proof and/or air tight control, for example, on fluid transfer conduit 60, further including venting capabilities in certain preferred embodiments. In certain embodiments, inner portion 10 and/or outer portion 20 may include insulated walls to further facilitate temperature control. In certain preferred embodiments, the ability to lower temperatures sufficiently to enable cryogenic storage capabilities may be provided, and may further provide a gradual warming capability to prepare the biological materials component of a biological chip device for use (e.g., for implantation).

The inner portion 10 of support container 8 may include any or all of the following, according to various embodiments of the invention:

A connection to the biological chip device (preferably water proof) that allows the biologic area to be exposed to a life-sustaining environment, while protecting the electronic components;

The connection may further provide MEMS power as well as feedback on conditions of the biologic component of the chip; this would allow for testing of both the circuit and biologic/cellular responses;

The connection to the biological chip device may further include a viral/bacterial filter, for example a high efficiency particulate air (HEPA) filter screen;

The support container 8 may have light emitters that can be adapted to test cellular response and allow for calibration of the biological chip device;

The support container 8 may be translucent in some embodiments; in certain preferred embodiments, it may have appropriate light filtering to prevent damage to cells from ultra-violet (UV) radiation, for example; and

The support container 8 and/or certain associated components may be adapted to be reusable, for example, by use of selected materials and design of components to enable sterilization and re-use.

Each support housing 6 may have its own power supply, which may also be adapted to be plugged in to an external source of power. Batteries, such as those used in medical and/or military applications, may be employed as power supplies. Batteries may be rechargeable, according to certain embodiments of the invention. For example, an AC adapter may allow a rechargeable battery to be recharged from a standard AC outlet via a transformer. In certain embodiments of the invention, charging or re-charging of the power supply for the biological chip device 30 may occur while in storage to preserve the power supply until needed.

In certain preferred embodiments of the invention, the support containers 8 may be reusable. Alternately, other embodiments of the invention may include disposable support containers 8. Reusable containers may include the ability to be hermetically sealed, including where access is needed for connections to the biological chip device 30. For example, a spring-loaded contact on the support housing 6 may allow for a hermetically-sealed “header” similar to those found on cardiac pacemakers and implantable defibrillators. Such a configuration would provide isolation of the support housing 6 from contaminants and pathogens, while allowing sterilization of the support housing 6 using standard techniques. The support housing 6 may further include disposable tubing that can supply separate isolated cooling, heating, and fluid/gas supply channels into and out of the support housing 6.

Support housing 6 should be designed to be rugged to withstand shock resulting from falls, etc. Containers may be made of any suitable material possessing requisite qualities, such as mechanical strength, heat tolerance, gas tolerance, and the ability to house both electronic componentry and biologic materials.

RFID technology can be used to keep track of the containers as well. The containers can be used as part of a network for transportation and delivery of the biological chip device to a hospital where the system can be tested to confirm cell viability. A device for testing may be portable (e.g., hand-held), and may interface with the container by available communication technology, such as blue tooth, or by directly plugging in to the support housing 6.

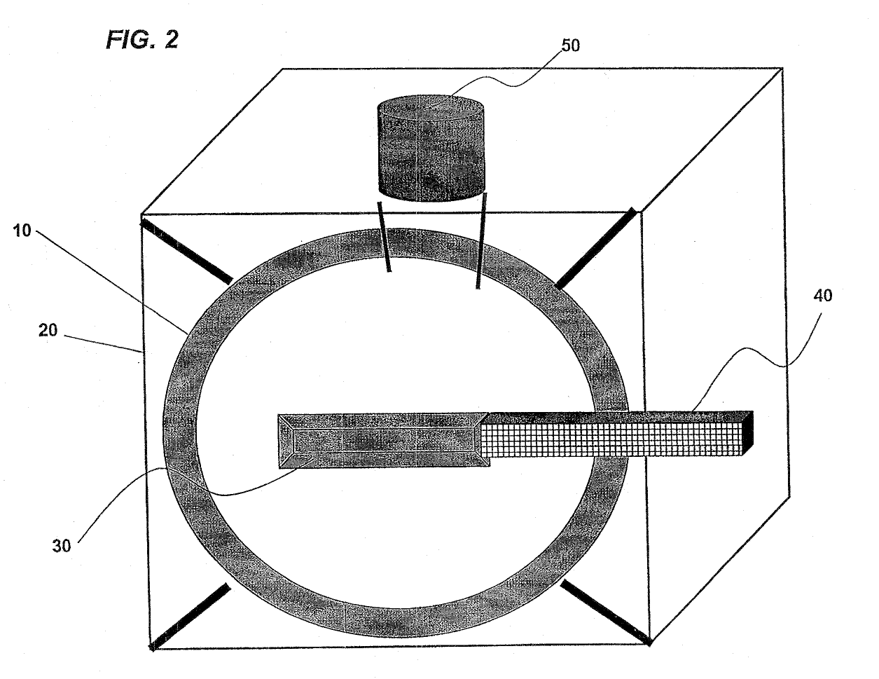

FIG. 2 is a schematic perspective view of a support housing 6 for a biological chip device according to an embodiment of the invention. In the embodiment shown in FIG. 2, a spherical inner portion 10 is shown disposed within a cubed (or rectangular) outer portion 20. This may be useful, for example, in applications that require the biological chip device to be at least partially immersed in a fluid substance. This configuration may also be useful in applications where enhanced control of temperature at various sections of the spherical container is desired. A spherical inner portion 10 may provide for equidistant photonic or detection arrays surrounding the biological chip device 30, and may provide added security and/or stability by placing the device near the center of the spherical inner portion 10. The 3D rectangular/cubical outer portion 20 surrounding the inner portion 10 according to this embodiment of the invention may facilitate convenient storage and positioning of the support housing 6, and may be useful in embodiments of the invention where a plurality of support housings 6 may be used.

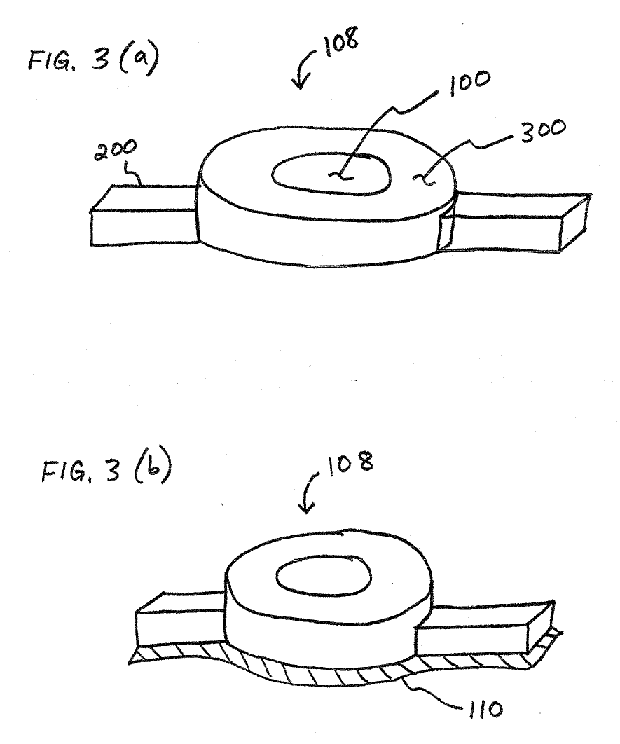

FIG. 3(a) is a schematic perspective view of an embodiment of the invention having an exemplary support container 108 comprising a first chamber 100 and a second chamber 300. First chamber 100 is where a living matrix chip 30 may be physically secured and/or environmentally sustained. Second chamber 300 may provide the ability to control the environment within first chamber 100. This may be done with electrical coils (i.e., for heating) and/or a fluid controlled system (for heating and cooling) disposed within second chamber 300.

FIG. 3(a) also shows input/output path(s) 200, which provide flowpaths for the flow of various services to and from the first chamber 100 and/or second chamber 300. For example, an input/output path 200 may contain electrical components and wiring needed to supply electrical power from a source external to support container 108 to a living matrix chip 30 disposed within first chamber 100. Input/output path 200 may also provide fluid flow to heating/cooling elements disposed within second chamber 300. For example, a “coolant” (e.g., chilled water, freon, etc.) may flow via tubing, for example, through input/output path 200 into the second chamber 300, where it absorbs heat from the first chamber 100, then is returned via input/output path 200 to an external cooling source. Liquid nitrogen, for example, may be used as a “coolant” for controlled cooling of the first chamber 100. In an embodiment utilizing liquid nitrogen, one or more pressure release valves (not shown) may additionally be required to control evaporation and address other pressure-related issues. One embodiment for long-term cryo storage may include the ability to continuously cycle liquid nitrogen through several networked support containers 108. The flow of nitrogen may be controlled by electronic valves adapted to supply the needed flow to the desired support containers 108 to achieve the desired temperature, for example.

Input/output path 200 may also be used to exchange fluid between the first chamber 100 and the environment external to the support container 108. Such fluid exchange may be necessary to provide a life-sustaining environment to the living matrix chip 30 disposed within first chamber 100, and/or may provide the ability to communicate information to or from the living matrix chip 30.

FIG. 3(b) shows a preferred embodiment of the invention having a vibration mount 110 coupled to the support container 108 to absorb physical shock (i.e., acceleration-related forces) and vibrations, and to thereby protect the living matrix chip 30 from damage that may be caused by such motion.

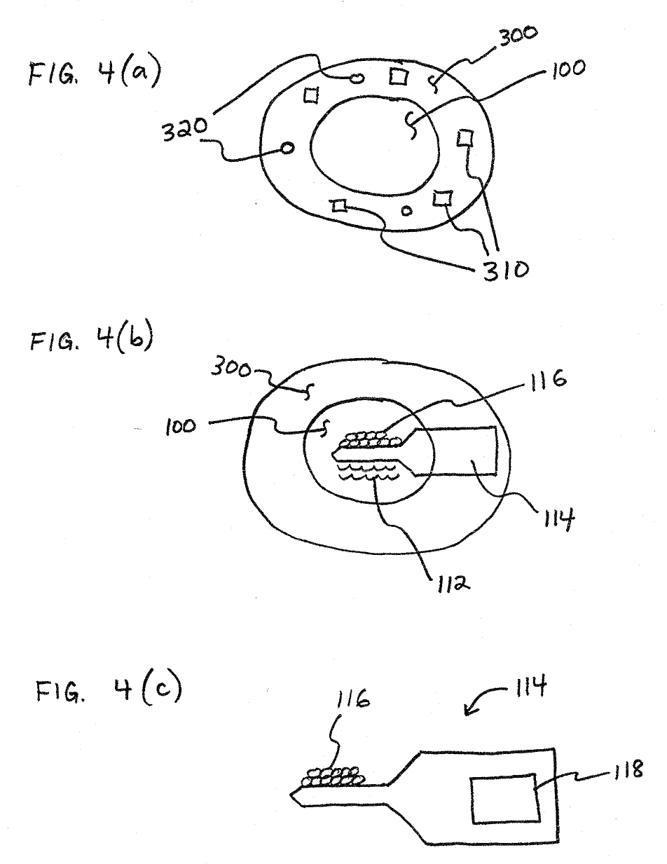

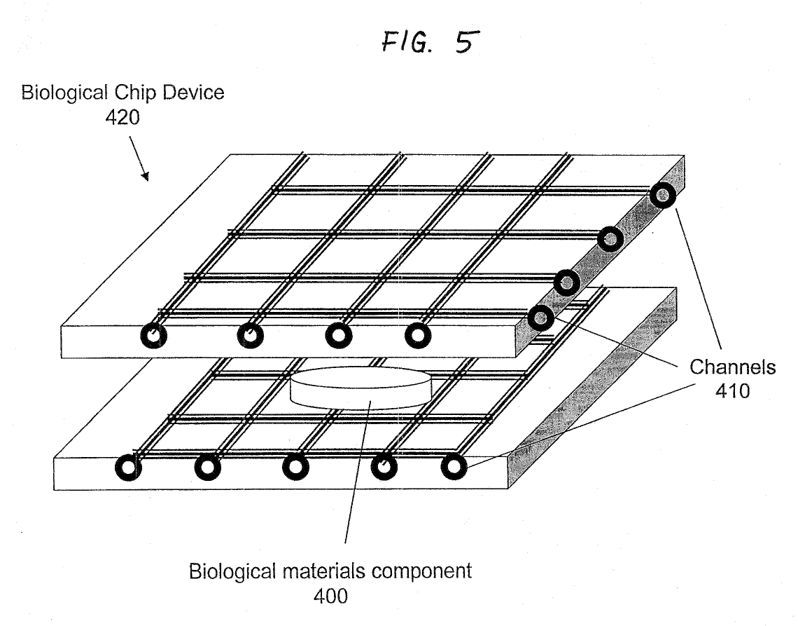

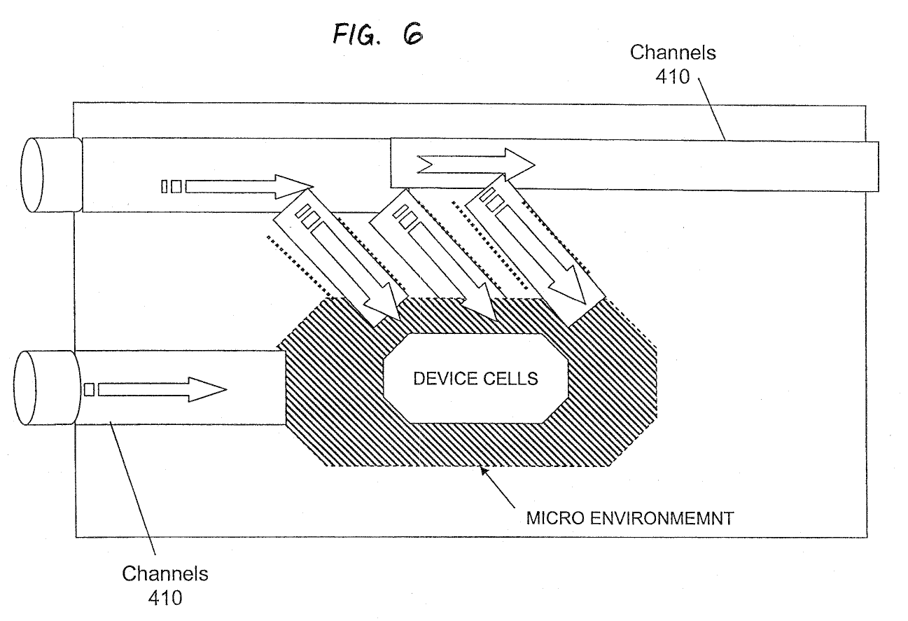

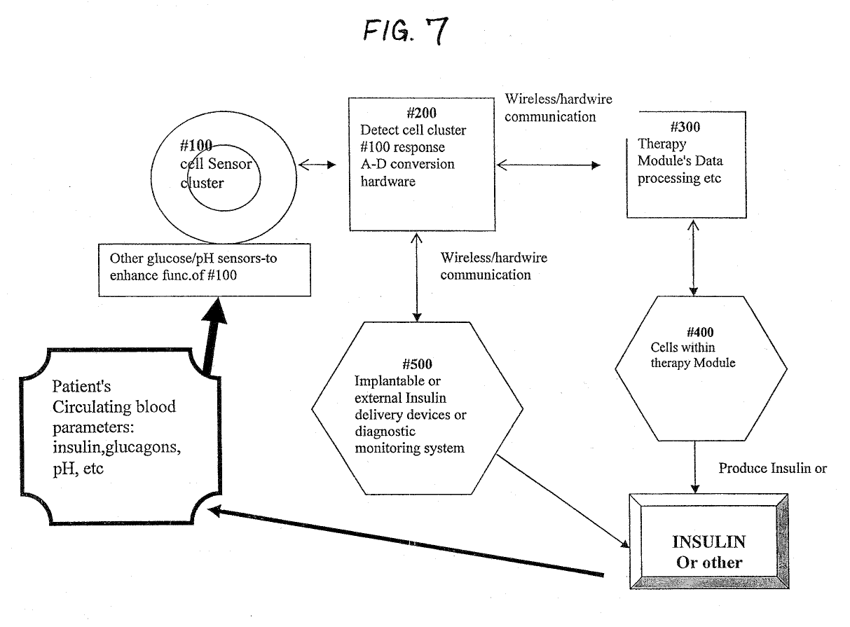

FIG. 4(a) is a schematic top plan view showing a possible arrangement of components in second chamber 300. For example, bio-sensors 310 may be disposed within second chamber 300 to monitor a number of environmental parameters, such as temperature and pH, for example and without limitation. Bio-sensors 310 may also include photodetectors, for example, to monitor levels of radiated energy, such as visible light, ultraviolet and infrared energy, and other forms of radiated energy.

Photo-emitters 320 may also be disposed within second chamber 300 in certain preferred embodiments of the invention. In one embodiment, photo-emitters 320 may emit light at known or specified wavelengths. The response of cells in the living matrix chip 30 may then be monitored using bio-sensors 310. For example, infrared energy at specific wavelengths may be emitted by photo-emitters 320, and the response of cells may be monitored by a bio-sensor 310 (such as a photodetector) to determine or confirm the continued viability of the cells in a given living matrix chip 30. Bio-sensors 310 may further include [enzymatic/chemical] and/or photo-receptor/detectors, and/or temperature/pH sensors, and/or light/laser-based means for detecting changes in cell shape, density, or visible alterations in the fluid/gaseous environment. Electrical sensors may be employed to detect static electricity and/or electromagnetic interference (EMI) that may potentially damage the chip. Other cell sensing may occur at the chip level and may be relayed back to the container electronics, and possibly to a computer or processor connected thereto.

Cell activity, cell growth, and modulation of the release of chemical or biological signals by cells may be controlled using a supply of fluids (e.g., certain drugs) and/or emission of electromagnetic energy (typically light energy) at various selected frequencies (e.g., by photo-emitters 320). Since certain cells may be designed and/or selected to produce fluorescent proteins, for example, cells may be adapted to respond to the presence of those proteins. This interaction between cellular materials, in addition to stimuli from light energy and/or fluid supply (e.g., chemical or drug) may be employed to block an activity, trigger an activity, or amplify a response, among other possible examples. This interaction could be controlled, for example, using various wavelengths of light, as well as combining it with infusion of drugs or other substances that work alone or in conjunction with the light. A specific wavelength or intensity of electromagnetic energy (e.g., light) may cause release of biologic or chemical substances that are initially “caged” or bound, but may be released when exposed to specific energies and wavelengths. The same electromagnetic energy can also enhance binding of biologic or chemical substances together to, for example, inhibit their activity or enhance their function by creating a link between two substances.

Substances, such as drugs, chemicals, proteins, etc., can also be infused into first chamber 100 to exist in a dormant, inactive state, but may be “triggered” by a certain stimulus (or by certain combinations of stimuli). For example, exposure of the substance to light energy, ultrasound energy, or RF energy (among many possible examples), may cause biological materials and/or chemicals stored in such substances to be released from their bound configurations and become “bio-available” to the matrix cells for nutrition, activation, and for other purposes.

One example of an exemplary technology that may be employed to supply the above-mentioned types of substances to the cells of a biological chip device is nanotube technology. Carbon nanotubes, for example, may be used for the above-described chemical and drug-elution processes. Additionally, carbon nanotubes may serve to provide structural support for cells within support container 8.

Nanotube technology can also be used in other portions of the support housing 6. For example, nanotubes can be used to form portions of fluid transfer conduit 60, such as the fluid pathways contained therein, or may form the conduit 60 itself. Nanotubes may be integrated into the wall of the piping or tubing between support apparatuses 6 to provide for controlled elution of drugs, chemicals, and/or other biochemical substances according to certain embodiments of the invention. Nanotube technology may thus facilitate flow-related release or delivery of chemical, drug, and biological substances, and may further facilitate the activated release of such substances using mechanisms (signals) such as heat, ultrasound, RF, magnetic field energy, and/or light energy stimulation. Alternatively, fluid flow from and to biological chip devices may be controlled (directed, shunted, bypassed, etc.) using valves, as well as circuitry that may be equipped with drug release mechanisms that may or may not use nanotube technology.

FIGS. 4(b) and 4(c) show an embodiment of the invention in which living cells 116 of a biological chip device are shown suspended and secured in first chamber 100 such that the living cells 116 are in contact with solution 112, which may be a saline, bio-supportive liquid, for example. As shown in FIG. 4(b), a support device 114 may physically support or suspend the living cells 116 in solution 112. The support device 114 may thereby provide protection to the living cells 116 from vibration, shock, or other forces. Further, support device 114 may provide the housing or support for electronics, electrical power, and communication signal pathways between the living cells and the external environment, as shown in the cross-sectional side view of FIG. 4(c).

A biologic-based sensor according to embodiments of the invention may include a detector for detecting the response of the cells to biological substances (e.g., to the level of glucose in the bloodstream). In one possible embodiment, a detector may be adapted to detect the level of fluorescent protein produced by the cells when the cells are exposed to glucose levels in the bloodstream. For example, the detector may have an optical component adapted to receive energy in the form of fluorescence (or bio-luminescence) from the cells. The detector may be coupled to (or may be an integral part of) a processor, such that the energy received at the detector may be used to generate diagnostic information related to the cell response. In a preferred embodiment, a detector and processor generate an information signal that estimates or indicates the amount of insulin that should be delivered to a patient based on the response of the cells to the glucose level in a patient's bloodstream.

The support housing may provide a means for exposing the cells to the biological substances. For example, the support housing may allow blood from a patient to come into contact with the cells within the housing to enable the cells to respond to the level of glucose in the blood, according to certain embodiments of the invention.

In some embodiments of the invention, the biologic materials component may include cells in which the genetic information has been modified using techniques known by those of ordinary skill in the art. For example, the insulin gene of the cells may be replaced by a fluorescent protein (e.g., green fluorescent protein, or “GFP”) to thereby produce an insulin-like protein that fluoresces and/or produces bioluminescence in response to the presence of glucose. The resulting fluorescence or bioluminescence may provide a measurable estimate or indication of the amount of insulin that should be delivered to the patient, according to certain embodiments of the invention.

Embodiments of the invention may include a device comprising an electronic component and a biologic materials component. The device may, for example, be adapted for use as an implantable medical device (IMD), and may form at least a portion of an IMD system. It may also be used as a stand alone diagnostic or research tool and not implanted.

For the treatment of a specific disorder which requires the detection and treatment of biological/biochemical substances, the device may use living cells that may be autologous and unaltered, or may be genetically engineered to detect and transmit the diagnostic data to: 1) a second biologic materials component (e.g., a second group of cells) that then provides a response to enhance the primary cell cluster's information, or to trigger a chemical response; or 2) an electronic component that converts the biologic cell response to digital/analog information which can be transmitted to other components of a medical device system; or combinations of both. In embodiments having two or more biologic materials components, the communications between the two cell clusters or matrices may occur within the same device or may be located on separate implanted devices. Communication between cell clusters and/or biologic materials components may occur via direct contact between the biologic materials, or via exchange of substances secreted by the cells, or by electrical stimulation of one cell matrix by another, or via an interface involving an electronics component, which may include, but is not limited to, optical or photonic detection of cell activity. Optical or photonic detection of cell activity, for example, may be translated to data and may provide a pre-determined output format for the receiving cell matrix.

A preferred embodiment of the invention may be useful in the treatment of diabetes. Pancreatic cells may be included as part of the biologic materials component of a device according to embodiments of the invention. Pancreatic cells may be grown into a matrix formation and sustained within a support housing as described herein. Pacreatic cells may be chosen to detect glucose levels, for example, and to provide a response that can be detected by: 1) a second group of cells; and/or 2) an electronics/photonics based detector system. In further embodiments, a device may use the information to deliver drug therapy to a patient, including the delivery of insulin or glucagon. Insulin delivery, for example, may be from an external insulin pump, an implantable insulin pump, or from a second portion of an implanted device that has a biological materials component adapted to produce insulin.

### EXAMPLE ONE

A glucose-sensitive cell line may be established using pancreatic cells, or other engineered cell lines that respond to glucose levels. The cells may respond to glucose levels by initiating a series of events that would normally lead to insulin production in normal pancreatic cells. However, in this example, the cells chosen produce fluorescent proteins rather than insulin. The protein produces a fluorescent signal, the intensity of which is related to the glucose level in the blood exposed to the cells. An additional embodiment may allow for the natural production of insulin tagged with fluorescent protein to provide a physiologically-based measure of the amount of insulin that should be administered. In other words, the more fluorescent tagged insulin (or insulin substitute) the cells create, the more insulin that should be delivered to address the existing glucose levels. This may provide for enhanced specificity and sensitivity in determining a treatment (e.g., an amount of insulin to administer to a patient) according to certain embodiments of the invention. Certain embodiments may allow for monitoring of both glucose and insulin levels, as well as the relationship between the two.

FRET technology may be employed to perform dual monitoring.

A non-photonic (or non-optical) based cell response detector method is described later in this application, and includes electrical, mechanical, passive photonic, and chemical reaction based forms of detection.

Architecture of cell embedded system: Pancreatic cells, or genetically modified pancreatic cells, or their equivalents, may be cultured onto an organized matrix with the required sensing/detector/electronics material.

Protection of biologic material on living chip from autoimmune responses and control of exposure of living chip to surrounding biologic environment: Cells or tissue used as part of the biologic materials component may be exposed to bodily fluids such as extra-cellular fluid, blood, etc. However, the biologic materials component should be protected from autoimmune responses within the person that can target the tissue. For example, certain autoimmune responses may target pancreatic cells. The support housing may be adapted to control the environment of the biologic materials component, which may include the ability to detect such autoimmune responses and/or prevent them from damaging the biologic materials component.

In certain embodiments, the biologic materials component may be shielded from the patient's antibody or autoimmune response using filter or tissue technology to neutralize and minimize contact between the device biologic material antigens and the surrounding environment.

Micro-Channel Fluid Control: Contact between cell clusters and the surrounding environment may also be controlled by using a series of hydraulic or hydrostatic channels which allowing only glucose, non-immune substances to be channeled to the biologic materials component and the remainder directed to an output channel on the device back into the surrounding tissue. These channels can also use electrostatic, charge dependent, pore limitation (e.g., channel size), or chemical structure (e.g., antibody based) binding and neutralization limiting mechanisms for control of core cell cluster exposure to surrounding environment.

In one embodiment of the invention, the biologic materials component cells may be arranged to provide for controlled exposure to the surrounding fluid environment. Oxygen, CO2, and other gases can be allowed to freely diffuse including glucose. A 3-dimensional configuration may be employed so that the biologic materials component of a biological chip device for managing diabetes is generally centered within the overall support structure; fluid channels may be disposed surrounding the biologic materials component. Antibodies, blood cells, etc., do not have direct contact with the cells. Substances that are similar to antibodies in size and cannot be excluded by limiting the diffusion barriers opening or pores can be selected further by organizing a series of channels in the device that sort and separate substances by electric charge (±), weight, binding to substances lining the channels and redirected to the output or bypass channels that lead back out of the biological chip device. The process for moving fluid and substances in and out of the channels can be based on natural hydraulic interactions with the surrounding tissues—one-way valves may facilitate movement through the device in a specific manner and direction. Devices that are implanted in areas of significant movement such as the abdomen, heart, lungs or muscles may operate by allowing natural hydraulic flow similar to that seen with siphoning fluid from one container to another, or the operation of a hand pump, wherein motion activates flow to “prime” the plumbing system. Additional help may come from the construction of several miniature pumps that prime the system on a periodic basis as needed to maintain flow.

The pumps may be built into the channels to inject solution or fluid from the surrounding environment into the channels or may employ transmission of pressure using a closed system that has fluid or gel inside that transmits mechanical pressure from natural movements to key portions of the channel system that is designed to be flexible and promote movement of fluid in a certain direction (e.g., channel pressure points).

Electrically driven pumps may be used in addition to the natural, hydraulic pumping as described above according to certain embodiments.

Electrically charged channels: The fluid channel walls may be electrically charged at various sections and assigned either a ± polarity. At points where separation is needed, positively charged substances may be guided toward one direction or channel X, the preceding channels may begin to be charged negatively as it approaches an intersection of channels (the Y). The channel leading in direction X (e.g., the desired direction) may be charged negatively to attract the positively charged substance. At this same section, the opposite walls of the channels leading toward another direction (the other path at the Y) may be charged positively in various intensities to simultaneously push the substance of interest (e.g., having a positive charge) away from the undesired path and toward the desired X direction or channel.

The charging of the channels can be accomplished by inserting electrodes throughout the channels or lining a portion of the internal channels near intersections to guide the substances in the right direction.

The charging and polarity can be controlled by an electronic component of the biological chip device, and may be changed as needed to enhance activity or movement according to certain embodiments of the invention.

Size limiting Channels: The channels can be designed to initially limit substances by size and exclude larger substances that are not needed for the biological chip device to function before such substances even enters the device channels. This can be done on an external surface of the channel opening and may be combined with electrically controlled charge features or channel pores coated with specific polymers that have intrinsic charge characteristics.

Size limitations can also be used within the device (e.g., in addition to the electrical charge system) to help separate substances.

Chemically mediated control of channel flow: Chemical substances that bind or neutralize unwanted chemicals or substances can be used to limit what is permitted to enter the biological chip device. This may include permanently fixed proteins that bind antibodies before they enter the device channels, and may include the coating of external aspects of the device that damage or neutralize antibodies directed to the specific cells within the device so that they are effectively prevented from entering the device channels.

The goal of the above-described fluid control mechanisms and methods is to minimize the effect of the body's immune response to the presence of an implanted biological chip device. It is accomplished in two ways: 1) by minimizing contact between the biologic materials component and the body's auto-immune system by shielding the cells' antigenic components from the biological chip device; and 2) if a reaction is initiated, minimize the potential for damage to the implanted device's biologic substrate.

Another embodiment of the invention may include a system which includes one or more biological chip devices adapted to produce a sensor response to a biological parameter, such as a blood parameter. Such a system may include an implantable medical device (IMD) or an external device (e.g., an insulin pump) which is adapted to communicate with the biological chip device, and which may be further adapted to deliver a treatment based upon a sensor signal received from the one or more biological chip devices. In certain preferred embodiments, the system includes a biological chip device adapted to produce a sensor signal that provides an indication of the amount of insulin that should be administered to a given patient.

Another embodiment of the invention may include a lead and/or lead system having a lead body and a biological materials component disposed thereon (e.g., at a distal end, or at multiple locations along the lead body). In a preferred embodiment, the biologic materials component may include pancreatic cells, or genetically modified pancreatic cells, or cells adapted to produce a response to glucose levels. The lead body may comprise one or more fiber optic strands adapted to communicate a cell response (such as a fluorescence signal) to a device, such as an implantable medical device, an external insulin pump, and/or a biologic chip device adapted to produce insulin, for example.

Another embodiment of the invention may include a method of treating a bio-chemical disorder such as diabetes. The following example illustrates a number of steps in a method that may be performed in accordance with an embodiment of the invention.

Description of Operation:

The device is separated into two primary components or areas of operations: a sensing module and a therapy module.

Steps:

Step 1: Once implanted into a patient, the diabetes living chip sensing modules #100 monitors the glucose levels. These cells may be pancreatic cell line that has been altered to produce a measurable response to glucose levels. This response may be the production of fluorescent protein instead of insulin.

Step 2: When glucose levels increase in the patient, the sensing module cells #100 detect the glucose increase within the intracellular space, which in turn triggers a cascade of events that would result in production of a fluorescent protein such as Green fluorescent protein (an example—other proteins are possible with various optical characteristics). The fluorescent response may directly correlate with the glucose level in one embodiment or may represent the physiologic insulin response that would have occurred if this was a normal insulin producing cell—this response would provide a measurement of the amount of insulin that is needed according the the sensing cell. The cell within the module may also consists of two cell types: one for each of the above functions or may use combined integrated proteins that give different fluorescence based on the binding in response to other intracellular substances.

Step 3: The fluorescence, or a substitute signal such as a chemical substance release, shape change, or electrical signal is detected by #200 which includes a photodetector and excitation array coupled to processing and conversion of the signal to data. For this example, we are using fluorescence as the measured response. The information is processed by #200 which is the electronics portion of the device directly responsible for #100 biologic materials and together form the Sensing module (100+200).

Step 4: Processed and unprocessed Data gathered by 200 may transferred via wireless, electrical, or photonic communication to either an implantable living chip therapy module #300, or implantable or external insulin delivery system #500, or a diagnostic implantable #501 or external monitoring device #502.

Step 5: For demonstration, the data obtained by Sensor module is communicated to therapy module data processing and control circuit #300 and decision made whether to provide therapy by producing insulin. The production of insulin is accomplished by the biologic material integrated into the therapy module #400.

Step 6: The cells within the therapy module #400 consists of insulin producing cells that do not need to be pancreatic in origin or even respond to glucose. These are genetically engineered cell lines that produce insulin in response to a selected trigger. This glucose sensing is not a requirement for these cells since sensing is accomplished by #100+200 and determination for therapy is decided y #300. The ability to separate therapy and sensing allows for flexibility in using the optimal cell types for each function.

Therapy Module Possible Configurations:

A) the therapy module cell line has intrinsic electrical depolarization characteristics such as that found in neuronal cells, cardiac, skeletal and vascular smooth muscle. The cells are engineered to produce insulin in response to electrical activation. Thus electrical field or pulses can be delivered to produce the needed insulin and control of the amount is determined by electrical amplitude ± frequency.

B) the therapy module cell lines produce insulin at baseline are negatively controlled using proteins available within the cytoplasm. The interactions of the se internal proteins can be controlled using photonic technology in the following possible configurations—

B1—the DNA gene sequence encoding insulin production is engineered to promote protein production occur only when a protein x is available. This protein X can consist of light responsive subcomponents that bind when exposed to specific wavelength light and are unbound at baseline. The reverse will also work, where the trigger protein is produced at baseline but inactive in the bound state. The active state is the unbound state that occurs when exposed to light.

B2—the cells produce insulin and the insulin gene is coupled to a specific receptor which can only be activated by secretion of a ligand/receptor agonist produced by a separate cell line that is integrated or mixed in with the insulin producing cells. Thus activation of the linking cells by light or electrical stimulation will produce release of a transmitter/agonist such as norepinephrine that then binds to the receptors of the insulin producing cells. This would trigger release of insulin.

In this scenario, the engineering of the molecular structure is more complex, m because it would require the design of two cell lines that interact with each other. It is possible to use neuronal cells to release substances such as norep quanta and have the norepinephrine molecule mutated so as to not stimulate or bind with high affinity to wild type or normal beta receptors. The beta receptor on the second cell type (insulin producing cell type) is also mutated to be specific for the norepi released by the associated neuronal cells within the living chip device. This would provide for specific communication and triggering between two different cell types each with different trigger mechanisms (neuronal—electrical and insulin producing-hormonal)

C) Insulin producing cells triggered by pulsatile vascular flow or repetitive movement in the body

These may be insulin producing cells that are engineered to be responsive to stretch, pressure and the periodicity of pressure/tension/stretch stimuli. When placed in the vascular system, they produce a steady state of insulin depending on the frequency, amplitude or the pulsations. Cells are selected based on their response to pulsatile blood flow. These cells can be used to maintain minimal baseline insulin levels with the ability to produce more when mechanically stimulated above the baseline physiologic rate.

In another application, the device can produce insulin on a substantially continuous basis and the insulin is collected in a separate reservoir for controlled release into the blood stream or surrounding subcutaneous tissue. The reservoir may be container which provides for survival of insulin and the balance of insulin which is active compared to inactive can be titrated. This reservoir can then mechanically released (open a port, pump it out, elution) the insulin into surrounding tissue in response to Sensing module or a linked therapy module.

What is claimed is:

1. A biologic-based sensor for treating a blood disorder in a patient, the sensor comprising:
cells capable of producing a response to changes in a blood parameter level;
a housing capable of sustaining the cells, the housing including
means for supplying nutrients and fluids to the cells being supported within the housing,
means for transferring energy to and from the cells being supported within the housing, and
means for removing waste products from the housing;
a detector for detecting the cell response; and
a processor coupled to the detector for generating diagnostic information from the cell response.

2. The sensor of claim 1 further comprising:
a therapy delivery subsystem in communication with the processor, the therapy delivery subsystem capable of delivering therapy to the patient in response to the diagnostic information.

3. A biologic-based sensor for treating a biochemical disorder in a patient, the sensor comprising:
cells capable of producing a response to certain biological parameters, the response being a function of the level of the biological substance exposed to the cells;
a housing capable of sustaining the cells, the housing including
means for supplying nutrients and fluids to the cells being supported within the housing,
means for transferring energy to and from the cells being supported within the housing, and
means for removing waste products from the housing;
a detector for detecting the cell response; and
a processor coupled to the detector for generating diagnostic information from the cell response.

4. The sensor of claim 3 further comprising:
a therapy delivery subsystem in communication with the processor, the therapy delivery subsystem capable of delivering therapy to the patient in response to the diagnostic information.

5. The sensor of claim 3 wherein levels of biological substances in a patient's bloodstream are monitored and diagnostic information produced therefrom.

6. The sensor of claim 4 wherein the biological substance is blood glucose.

7. The sensor of claim 3 wherein levels of biological substances in a patient's organs are monitored and diagnostic information produced therefrom.

8. The sensor of claim 3 wherein levels of biological substances in a circulatory system of a patient are monitored and diagnostic information produced therefrom.

9. A system for monitoring and treating bio-chemical disorders, the system comprising:
a biological chip device having a biologic materials component, the biological chip device adapted to sense a physiological parameter from a patient and provide diagnostic information; and
a medical device adapted to receive diagnostic information from the biological chip device, and further adapted to delivery a therapy to the patient in response to the diagnostic information.

10. A lead for monitoring a bio-chemical disorder, the lead comprising:
a lead body; and
a biological chip device disposed along the lead body, the biological chip device having a biologic materials component, the biological chip device adapted to sense a physiological parameter from a patient and communicate diagnostic information via the lead body.

11. A method of treating a biochemical disorder substantially as described above.
